(19) United States
(12) Patent Application Publication
(10) Pub. No.: US 2012/0100211 A1
(43) Pub. Date: Apr. 26, 2012

(54) MATERIAL AND PROCESS FOR INCORPORATION OF LOW DOSAGE ACTIVE PHARMACEUTICAL INGREDIENTS AND USE THEREOF

(76) Inventors: James Farina, Nazareth, PA (US); Nandu Deorkar, Cedar Knolls, NJ (US); Liliana Miinea, Easton, PA (US)

(21) Appl. No.: 13/261,010

(22) PCT Filed: Jul. 12, 2010
(86) PCT No.: PCT/US10/41665

§ 371 Date: Nov. 14, 2011

Related U.S. Application Data
(60) Provisional application No. 61/225,942, filed on Jul. 16, 2009.

(51) Int. Cl. A61K 9/20; A61K 9/28; A61K 9/14

(52) U.S. Cl. 424/465; 424/490; 427/2.14

(57) ABSTRACT

A low dose API pharmaceutical tablet having excellent content uniformity is provided. The tablet is formed by spray coating a support excipient with the API. The resulting composition is suitable for direct compression tablet formulation without the need for an additional granulation step to uniformly coat the API onto the support excipient. The support excipient comprises microcrystalline cellulose, a binder and a disintegrant, and is formed by spraying a homogeneous slurry of the support excipient components.

### BACKGROUND OF INVENTION

Pharmaceutical tablets are the most commonly employed means to deliver drug substances. A primary difficulty in the formulation and manufacturing of low dose active pharmaceutical ingredients (API) is product content uniformity. Additionally, decrease in the drug potency due to manufacturing losses, as well as tablet instability due to the increased ratio of excipients to the API, are observed at low API dosages.

At low dosages, good content uniformity, or blend uniformity of an API with the excipient during the blend process is challenging, especially where there are significant differences between the excipient and API particle size and shape. Efficient mixing becomes a significant issue when the API represents less than about 5% of the total mixture, and more critical when the API content is below about 1%. Drug uniformity tests of powder blends and finished dosage units such as tablets, as well as dissolution tests are tools used to determine whether the active ingredient is evenly distributed in the blend and each resulting tablet. FDA regulations (FDA: Guidance for Industry: Powder Blends and Finished Dosage Units—Stratified In-Process Dosage Unit Sampling and Assessment, October 2003) state that the information submitted to support an application for a drug must include in-process tests, such as blend uniformity analysis, useful for ensuring the adequacy of the mixing of API with other components of the drug product. Batch uniformity analysis is recommended for those drug products for which the USP requires content uniformity analysis. USP requires this test when drug products contain less than 50 milligrams of the API per dosage form unit, or when the API is less than 50% of the dosage form unit by weight. Acceptance criteria of 90.0% to 110.0% of the expected quantity of API, with a relative standard deviation of no more than 5.0%, are recommended for batch uniformity analysis.

Conventional methods used to provide sufficient uniformity include the use of dry or wet granulation technology. This additional step increases the cost and time of production. An attempt to improve the process of incorporating a low dosage of API into a solid dose form was disclosed by Wan, Heng, and Muhurri (International Journal of Pharmaceutics, 88 (1992) 159-163). The authors attempted to improve the spray granulation process of incorporating a low dosage API by spraying the drug into the mixture during a spray granulation step. It was purported that mixing the API with a binder during the spray granulation process resulted in improved blend uniformity. However, this method still requires the additional granulation step. There exists therefore a need for a material and a process to produce tablets with a very low dose of API (less than about 5%) that can facilitate use of direct compression without an additional dry or wet granulation step.

### SUMMARY OF INVENTION

An illustrative aspect of the present invention is a composition comprising a) a support excipient comprising: about 75% to about 98% microcrystalline cellulose (MCC); about 1% to about 10% at least one binder; and about 1% to about 20% at least one disintegrant; wherein the microcrystalline cellulose, binder and disintegrant are indistinguishable when viewed with a scanning electron microscope (SEM), thereby forming substantially homogeneous, substantially spherical particles of the support excipient; and b) an API spray coated on the support excipient, wherein the API is about 0.01% to about 5% by weight of the support excipient.

Another illustrative aspect of the present invention is a method of making a low dose API tablet, the method comprising: a) forming a support excipient by mixing a MCC slurry with a disintegrant slurry to form a MCC/disintegrant slurry; mixing a binder in water to form a viscous binder slurry; homogenizing the binder slurry with the MCC/disintegrant slurry to form a homogenized slurry; and spray dry granulating the homogenized slurry to form substantially homogeneous, substantially spherical particles of support excipient; b) spray coating the support excipient with less than about 5% of an API; and c) drying the spray coated support excipient to form API coated support excipient particles. A further illustrative aspect of the present invention is a low dose API pharmaceutical tablet comprising about 0.01% to about 5% of at least one active pharmaceutical ingredient; and an support excipient of substantially homogeneous, substantially spherical particles including: microcrystalline cellulose; at least one binder; and at least one disintegrant.

### BRIEF DESCRIPTION OF DRAWINGS

FIG. 1 is an illustration of SEM micrographs of the support excipient produced according to Example 1.

FIG. 2 is an illustration of SEM micrographs of the support excipient produced according to Example 2.

### DETAILED DESCRIPTION

There is provided a composition and method that provide a low dose API coated support excipient. The coated support excipient is suitable for direct compression tablet production without the use of an additional granulation step, and provides excellent content uniformity.

In a preferred illustrative embodiment the support excipient is combined with the low dosage API using a spray coating process, for example Wurster coating, in a fluidized bed. The low dosage API can be spray coated onto the support excipient in the range of about 0.01% to about 5% while maintaining sufficient content uniformity. In a more preferred illustrative embodiment the API is about 0.01% to about 2%. All percentages disclosed and claimed are weight/weight percentages unless otherwise noted. API percentages are percentages of the API in a completed solid dosage form, unless otherwise noted.

The support excipient comprises substantially homogeneous, substantially spherical particles of a highly compressible granular microcrystalline cellulose based support excipient, which is the subject matter of U.S. Provisional Patent Application Ser. No. 60/978,866, incorporated herein by reference. As defined herein, the term ‘substantially homogenous particles’ is defined as a composition in which the individual components of the composition are not individually distinguishable when viewed under SEM.

The support excipient has strong intraparticle bonding bridges between the components, resulting in a unique structural morphology including significant open structures or hollow pores. The presence of these pores provides a surface roughness that is the ideal environment for spraying with an API. Additionally, this support excipient contains a binder and a disintegrant which in the process of coating absorb water and bind the drug to the particles. Additionally, this support excipient includes the necessary excipients, except for the optional lubricant, that are required to produce a pharmaceutically acceptable tablet.

The support excipient is engineered to have particle size that results in the support excipient being directly compressible, complete, and universal excipient for making pharmaceutical tablets. The support excipient is considered complete since it includes a diluent, a binder and a disintegrant, and universal since it is surprisingly compatible with a variety of APIs. The components and physical characteristics of the support excipient were carefully chosen and optimized to ensure its use in formulating a wide range of APIs.

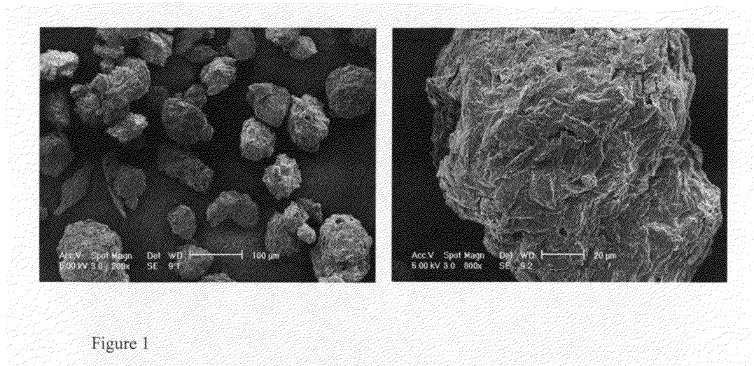

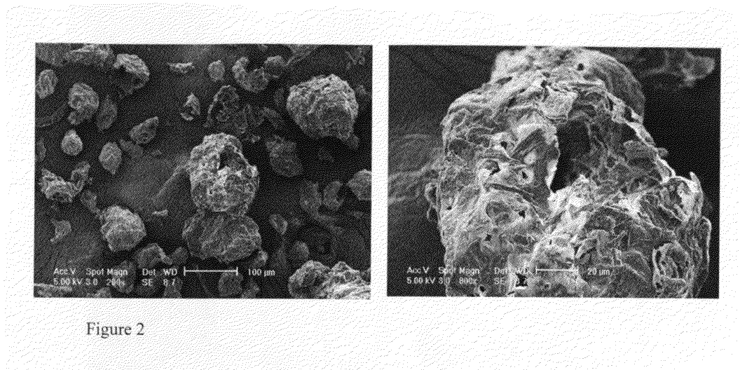

In the present invention, MCC is processed in combination with a polymeric binder and a cross-linked hygroscopic polymer disintegrant to produce spherical particles, having high porosity and strong intraparticle binding. The polymeric binder is selected from the class of cellulosic polymers or organic synthetic polymers having thermal stability at about 80° C. to about 120° C., dynamic viscosity in the range of about 2 mPa to about 50 mPa for a water solution of about 0.5% to about 5% wt/vol, water solubility in the range of about 0.5% to about 5% wt/vol and providing a surface tension in the range of about 40 dynes/cm to about 65 dynes/cm for about 0.5% to about 5% wt/vol water solution. Preferred binders from this class include hydroxypropyl methylcellulose, hydroxyethyl cellulose, hydroxypropyl cellulose, sodium carboxymethyl cellulose, and polyvinyl alcohol-polyethylene glycol graft copolymer, vinylpyrrolidone-vinyl acetate copolymer and mixtures thereof. Presently preferred is hydroxypropyl methylcellulose (HPMC). The cross-linked hygroscopic polymer disintegrant is preferably crospovidone (CPVD). As is seen in FIGS. 1 and 2, the processed particles are a substantially homogeneous composition of spheres with porous portions leading to at least partially hollow portions of the spheres. The particles are produced by the actual physical binding of the slurry mixture that becomes distinct particles when ejected out of the nozzle. The porosity and hollow portions result in improved API loading and blendability.

The process disclosed herein is a novel form of the spray drying granulation process. The new process consists of the homogenization of all three components of the support excipient in the presence of water to create a slurry of the components. In one non-limiting, illustrative embodiment, a slurry of MCC is mixed with a slurry of cross-linked polyvinylpyrrolidone slurry to form a MCC/cross-linked polyvinylpyrrolidone slurry. Hydroxypropyl methylcellulose is then mixed with water to form a viscous hydroxypropyl methylcellulose slurry. The hydroxypropyl methylcellulose slurry is then mixed/homogenized with the MCC/cross-linked polyvinylpyrrolidone slurry to form a homogenized slurry. The homogenized slurry is then spray dry granulated to form substantially homogeneous, substantially spherical particles of support excipient. It is noted that this granulation process does not include the API.

The homogenization process is carried out to bring the two insoluble components, MCC and a disintegrant, in contact with each other and bound in close association with a viscous binder solution, for example hydroxypropyl methylcellulose. The evaporation of water at a high rate at high temperatures of 120° C. or more and the local action of HPMC holding all components together produces particle with unique shape and morphology.

The components of the support excipient are processed by an improved wet homogenization/spray dry granulation method. In this process, a slurry is formed of two water insoluble components (typically with a large difference in composition between the two water insoluble components) and a third water soluble component. The resulting slurry is granulated to a desired particle size, typically greater than about 50 μm, preferably about 50 μm to about 250 μm, and more preferably about 90 μm to about 150 μm.

The support excipient is formed by processing, or homogenizing, MCC with the polymeric binder and a cross-linked hygroscopic polymer disintegrant. In an illustrative embodiment, the support excipient is formed from about 75% to about 98% MCC, in combination with about 1% to about 10% binder and about 1% to about 20% disintegrant. In a preferred embodiment, the support excipient is formed from about 80% to about 90% MCC, about 2% to about 8% binder and about 3% to about 12% disintegrant. In a more preferred embodiment, the support excipient is formed from about 85% to about 93%, about 2% to about 5% binder and about 10% disintegrant. Illustrative formulations of the support excipient are given in Examples 1 through 3. All percentages herein are wt/wt unless otherwise noted.

The “active ingredient” or “active agent”, referred herein as the API, refers to one or more compounds that have pharmaceutical activity, including therapeutic, diagnostic or prophylactic utility. The pharmaceutical agent may be present in an amorphous state, a crystalline state or a mixture thereof. There is no limitation to the active pharmaceutical ingredient (API) that can be used with the present invention except that in which the API is incompatible with the microcrystalline cellulose.

Illustrative suitable active ingredients that can be used with the present invention include, but are not limited to: opioid analgesics, including but not limited to hydromorphone hydrochloride; ACE inhibitors including but not limited-to trandolapril, lisinopril, ramipril; immunosuppressant, including but not limited to tacrolimus; muscarinic receptor antagonist including but not limited to solifenacin succinate; angiotensin II receptor antagonist including but not limited to candesartan cilexetil; calcium channel anatgonist including but not limited to felodipine; non-steroidal aromatose inhibitor including but not limited to anastrozole; alpha-agonist hypotensive agent including but not limited to clonidine hydrochloride; dopamine agonist including but not limited to pramipexole dihydrochloride; synthetic vitamin D analogue including but not limited to doxercalciferol; anticoagulant including but not limited to warfarin sodium; antiepylepsy agent including but not limited to tiagabin hydrochloride; acetylcholinesterase inhibitor including but not limited todonepezil; hormones including but not limited to thyroid, liotrix, levothyroxine; antidiabetic agent including but not limited to rosiglitazone maleate; cardiac glycoside including but not limited to digoxin; human neoplastic disease including but not limited to chlorambucil; serotonine 5HT3 receptor antagonist including but not limited to alosteron hydrochloride; non-ergoline dopamine agonist including but not limited to ropinirole hydrochloride; psychotropic agent including but not limited to risperidone, olanzapine; synthetic adrenocortical steroid including but not limited to dexamethasone; central nervouse system stimulant including but not limited to dexmethylphenidate, alprazolam; blood glucose lowering drug including but not limited to rapaglimide, glimepiride; calcium ion antagonist including but not limited toamlodipine besylate; antiseizure and antipanic including but not limited to clonazepam; antiemetic including but not limited to granisetron hydrochloride; hypnotic agent including but not limited to eszoplicone.

The incorporation of a low-dosage API in a formulation can be accomplished by any conventional spray coating procedure. A suitable, illustrative method is Wurster coating. In this process the product is fluidized upward into an expansion chamber. The product passes through a spray zone where an atomized spray coating solution is applied. The Wurster coater uses a draft tube suspended above the spray nozzle at the bottom of the apparatus. The particles flow inward, under the edge of the draft tube, from the annular region outside the draft tube. They are partially coated as they move upward in the draft tube, propelled by higher airflow as well as by air from the fluid nozzle. After moving upward, drying in the air, the particles fall back and make a return trip to the nozzle where they receive more coating. This gives more uniform coating. The Wurster process gives less erosion of the particles and very good coating uniformity.

The API may be spray coated onto the support excipient (MCC/HPMC/CPVD) by several methods to form API coated support excipient particles. Suitable methods include spray coating the support excipient with a solution of the API; spray coating the support excipient with a slurry of a micronized API; or spray coating the support excipient with a low solubility API solution containing an API dissolution aid.

The support excipient includes both a binder and a disintegrant, and is therefore considered a ‘high functionality’ excipient, given that only an optional lubricant need be added before direct compression. It is noted however, that additional excipients, for example glidants, may be added if desired, as is well known in the art.

Non-limiting examples of the present process and composition are illustrated in the Examples. Examples 1, 2 and 3 disclose non-limiting examples of the production of suitable support excipients. Compositions of the support excipient, Wurster coated with 1% and 0.1% Chlorpheniramine Maleate are shown in Examples 4 and 8 respectively. The 1% loading provided a content uniformity of 6.06 and the 0.1% loading provided a content uniformity of 5.89.

Examples 5, 6 and 7 illustrate that a combination of the support excipient, formulated as disclosed, and spray coating of the API is necessary to achieve the content uniformities achieved in Examples 4 and 8. In Example 5, 1% chlopheniramine maleate was physically mixed with the support excipient, rather than spray coated as in Example 4. The resulting product uniformity was 18.83. Example 6 utilizes the components of the support excipient, but the components were merely physically mixed as opposed to being formulated as disclosed herein. The physically mixed excipient was spray coated with the API resulting in a product uniformity of 17.95. In Example 7, excipient components and the API were physically mixed, resulting in a product uniformity of 42.97. It has therefore been shown that both the support excipient, prepared as disclosed herein, and API spray coating thereon are required to attain sufficient content uniformity.

Example 9 discloses a composition according to the present invention wherein the support excipient is spray coated with Ibuprofen at 1% drug loading. An excellent drug uniformity, as determined by percent relative standard deviation (% RSD), was observed to be 4.1. For comparison, in Example 10, the support excipient was physically mixed with 1% ibuprofen, resulting in a content uniformity of 15.99.

Example 11 discloses a composition of 1% Hydrochlorotiazide and support excipient prepared by a conventional method used to apply low dosages of API, namely geometric dilution method. This example illustrates again that the direct mixing of the support excipient with a low dosage drug results in poor content uniformity of the drug in the powder blend. The poor content uniformity may be influenced by the large differences between the particle size of the support excipient and the API, and/or by API loss on the walls of the blender during the blending process.

It is therefore disclosed that the unique properties of the instant support excipient, prepared as disclosed herein, in combination with the spray coating process, can be used to successfully incorporate a low dosage API onto the support excipient for making tablets without an additional wet granulation step, for example by direct compression. The tablets prepared in the Examples by direct compression have excellent hardness and disintegration times as illustrated in Example 12. The tableting by direct compression of the low dosage API spray coated on the support excipient will typically require only the addition of a lubricant.

### Examples 1-3

### Methods of Making the Support Excipient

### Example 1

### Preparation of Microcrystalline Cellulose-2% Hydroxypropyl Methylcellulose—Crospovidone Support Excipient According to the Present Invention

The support excipient consists of microcrystalline cellulose at 85%, hydroxypropyl methyl cellulose at 2%, and crospovidone at 13%. The support excipient was produced by a wet homogenization/spray dry granulation process. The apparatus used for the production of the support excipient was a Co-current atomizer disc type with the disc RPM between 12000 and 25000 and the inlet temperatures of 180-250° C. Powdered MCC was converted into a slurry in a mixing chamber with deionized water to give a concentration of 23.3%. The other components, HPMC and crospovidone were also converted to a slurry with deionized water in a separate mixing chamber at 60° C. to a concentration of 5.9%. The MCC slurry was then transferred to the chamber containing the HPMC/crospovidone slurry and homogenized into a uniform mixture at 40-60° C. for 1 hour using circulating shear pump and an agitator to keep solids suspended in the solution thereby forming a uniform slurry. The slurry mixture was then spray dried through a rotary nozzle at a motor frequency of 33 Hz in the presence of hot air at an outlet temperature of 106-109° C. This constitutes the particle formation step. The fines were removed in a cyclone and the final product was collected to give the new support excipient. SEM micrographs of the support excipient of Example 1 are seen in FIG. 1. Unless otherwise noted, all SEM micrographs herein were recorded using a FEI XL30 ESEM (environmental scanning electron microscope), voltage 5 kV, spot size 3, SE detector. The samples were sputtered with Iridium before SEM analysis (sputtering time 40 sec.)

The compressibility, aerated bulk density and tapped bulk density of the support excipient were measured using a Powder Tester (Hosokawa Micron Corporation) Model PT-S. A computer which uses the Hosokawa Powder Tester software was used to control the Hosokawa Powder Tester during the measurement operation, enabling simple use and data processing. For measuring the aerated bulk density and tapped bulk density a 50 cc cup was employed. The standard tapping counts for measuring the tapped bulk density were 180 and the tapping stroke was 18 mm. D50 value was calculated based on the data collected in a “particle size distribution” measurement. An Air Jet Sieving instrument (Hosokawa Micron System) was used to determine the particle size distribution of the support excipient. A set of four sieves (270 mesh, 200 mesh, 100 mesh and 60 mesh) was used. The sieving time for each sieve was 60 sec, while the vacuum pressure was maintained at 12-14 in. H₂O. The sample size was 5 g.

The “loss on drying” (LOD) value was determined using a Mettler Toledo Infrared Dryer LP 16. The set temperature was 120° C. and the analysis was stopped when constant weight was reached.

TABLE 1

| Powder Characteristics | Value |
|---|---|
| 1. Compressibility | 16.1% |
| 2. D50 | 113 um |
| 3. Aerated bulk density | 0.29 g/cc |
| 4. Tapped bulk density | 0.35 g/cc |
| 5. LOD | 3.0% |

### Example 2

### Preparation of Microcrystalline Cellulose-5.5% Hydroxypropyl Methylcellulose—Crospovidone Support Excipient According to the Present Invention

The support excipient consists of microcrystalline cellulose at 85.5%, hydroxypropyl methyl cellulose at 5.5%, and crospovidone at 9%. The support excipient was produced by a wet homogenization/spray drying granulation process. The apparatus used for the production of the support excipient is a Co-current atomizer disc type with the disc RPM between 12000-25000 and the inlet temperatures of 180-250° C. After granulation a cyclone separation device was used to remove the fines. Powdered MCC was converted into a slurry using deionized water in a mixing chamber to reach a concentration of 25.1%. The other components HPMC and crospovidone were first dry mixed and then also converted into a slurry with deionized water in a separate mixing chamber to a concentration of 11.4%. The MCC slurry was then transferred to the chamber containing the HPMC/crospovidone slurry and homogenized into a uniform mixture at 40-60° C. for 1 hour using circulating shear pump and an agitator to keep solid suspended in the solution to form uniform slurry The slurry mixture was then spray dried through a rotary nozzle at the motor frequency of 40.1 Hz in the presence of hot air at an outlet temperature of 106-109° C. This constitutes the particle formation step. The fines were removed in a cyclone and the final product was collected, see FIG. 2.

The powder characteristics were determined as described in example 1.

TABLE 2

| Powder Characteristics | Value |
|---|---|
| 1. Compressibility | 19.7% |
| 2. D50 | 104 um |
| 3. Aerated bulk density | 0.20 cc/g |
| 4. Tapped bulk density | 0.25 cc/g |
| 5. LOD | 2.0% |

### Example 3

### The Support Excipient Consists of Microcrystalline Cellulose at 89%, Hydroxypropyl Methyl Cellulose at 2%, and Crospovidone at 9%

The support excipient was produced by a wet homogenization/spray drying granulation process. The apparatus used for the production of the support excipient was a Co-current atomizer disc type with the disc RPM between 12000-25000 and the inlet temperatures of 180-250° C. After granulation a cyclone separation device was used to remove the fines. The production of the support excipient begins with converting powdered MCC (which consists of rod like particles) into a slurry using deionized water in a mixing chamber to a concentration of 23.3%. In a separate container corspovidone was added to deionized water to form a 12.4% slurry. In another tank HPMC was added to deionized water to form a 7.3% slurry. One third of the MCCc slurry was transferred in a mixing tank and ⅖ of the crospovidone slurry was added to it under continuous stirring. This step was repeated until all the MCC and CPVD slurries were mixed together. The MCC/CPVD slurry was homogenized for 75 min. To the MCC/CPVD slurry was added the HPMC slurry and the final mixture was homogenized for 75 min. During the whole mixing process the homogenization is performed using a circulating shear pump and agitator. The resulting slurry mixture was then spray dried through a rotary nozzle at the motor frequency of 32.5 Hz in the presence of hot air at an outlet temperature of 106-109° C. This constitutes the particle formation step. The fines were removed in a cyclone and the final product was collected.

The powder characteristics were determined as described in example 1.

TABLE 3

| Powder Characteristics | Value |
|---|---|
| 1. Compressibility | 16.5% |
| 2. D50 | 117 um |
| 3. Aerated bulk density | 0.27 g/cc |
| 4. Tapped bulk density | 0.34 g/cc |
| 5. LOD | 5.7% |

### Example 4

### Wurster Coating of 1% Aqueous Solution of Chlorpheniramine Maleate on Support Excipient

Wurster coating was carried out in a Fluid Air bench top unit using a 2 L bowl and the chamber extension. The partition gap was set to ¾ inch. 495 g of support excipient were loaded in the bowl. The air flow rate was set to an optimum value of 12 SCFM. The inlet temperature was maintained at 80° C. 100 mL of aqueous solution containing 5 g of chlorpheniramine maleate was delivered at a rate of 3.8 mL/min using an atomizing air pressure of 15 psi. During coating the product temperature varied between 40 and 50° C. After all the drug solution was sprayed the product was dried at 45-50° C. maintaining an air flow rate of 10-12 SCFM until a LOD value of ˜1% was achieved. The material was discharged from the system and analyzed for chlorpheniramine maleate content uniformity.

Chlorpheniramine Maleate Content Uniformity Analysis:

The powder was poured on a flat surface and divided in eight parts. Three powder samples of about 200 mg each, were taken and placed in a separate flask. 100 mL of 0.01 N HCl was added to each sample. The vials were shaken periodically. After 24 hours samples were taken from each vial, filtered through a 45 μm nylon filter and then analyzed by UV-Vis. The amount of chlorpheniramine maleate in each sample was calculated by employing the UV absorption at λ=265 nm, the wavelength of maximum absorbance for chlorpheniarmine maleate. Standards were prepared in 0.01N HCl. A correction for the support excipient absorbance at 265 nm was applied when calculating the amount of chlorpheniramine maleate in each sample. The content uniformity was very good, with a 6.06% RSD.

### Example 5

### Preparation of a Blend of 1% Chlorpheniramine Maleate in Support Excipient by Blending in a V-Blender

99.0 g of support excipient were mixed together with 1.0 g of chlorpheniramine maleate in a V-blender for two hours. The blend was discharged and analyzed for content uniformity using an equivalent method with the one described in Example 4. The content uniformity analysis gave a % RSD of 18.83.

### Example 6

### Wurster Coating of 1% Aqueous Solution of Chlorpheniramine Maleate on a Blend of MCC, HPMC and CPVD

A blend of 85.5 g microcrystalline cellulose, 5.5 g hydroxypropyl methylcellulose and 9 g crospovidone was prepared by blending in a V-blender for two hours. The MCC, HPMC, CPVD blend was then loaded in the Wurster coater bowl. Wurster coating was carried out in a Fluid Air bench top unit using a 2 L bowl. The air flow rate was set to an optimum value of 12 SCFM. The inlet temperature was maintained at 60° C. 100 mL of aqueous solution containing 1 g of chlorpheniramine maleate was delivered at a rate of 4 mL/min using an atomizing air pressure of 5 psi. During coating the product temperature varied between 40 and 50° C. After all the drug solution was sprayed the product was dried at 45-50° C. maintaining an air flow rate of 10-12 SCFM until a LOD value of ˜1% was achieved. The material was discharged from the system and analyzed for chlorpheniramine maleate content uniformity using an equivalent method with the one described in Example 4. The content uniformity analysis gave % RSD of 17.95.

### Example 7

### Preparation of a Blend of 1% Chlorpheniramine Maleate in MCC/HPMC/CPVD by Blending in a V-Blender

A blend of 85.5 g microcrystalline cellulose, 5.5 g hydroxypropyl methylcellulose and 9 g crospovidone was prepared by blending in a V-blender for two hours. To the resulting blend 1 g of chlorpheniramine maleate was added and the mixture was blended in a v-blender for two hours. The blend was discharged and analyzed for content uniformity using an equivalent method with the one described in Example 4. The content uniformity gave a % RSD of 42.97.

### Example 8

### Wurster Coating of 0.1% Aqueous Solution of Chlorpheniramine Maleate on the Support Excipient

Wurster coating was carried out in a Fluid Air bench top unit using a 2 L bowl and the chamber extension. The partition gap was set to ¾ inch. 499.5 g of support excipient were loaded in the bowl. The air flow rate was set to an optimum value of 12 SCFM. The inlet temperature was maintained at 80° C. 100 mL of aqueous solution containing 0.5 g of chlorpheniramine maleate was delivered at a rate of 3.8 mL/min using an atomizing air pressure of 15 psi. During coating the product temperature varied between 38 and 42° C. After all the drug solution was sprayed the product was dried at 45-50° C. maintaining an air flow rate of 10-12 SCFM until a LOD value of ˜2% was achieved. The material was discharged from the system and analyzed for chlorpheniramine maleate content uniformity (see below). The content uniformity was very good, with a % RSD of 5.89.

### Example 9

### Wurster Coating of 1% Aqueous Solution of Ibuprofen on the Support Excipient

Wurster coating was carried out in a Fluid Air bench top unit using a 2 L bowl. 100 g of support excipient were loaded in the bowl. The air flow rate was set to an optimum value of 12 SCFM. The inlet temperature was maintained at 60° C. 75 mL of aqueous solution containing 1 g of Ibuprofen in 0.1N NaOH was delivered at a rate of 4 mL/min using an atomizing air pressure of 3 psi. During coating the product temperature varied between 40 and 50° C. After all the drug solution was sprayed the product was dried at 45-50° C. maintaining an air flow rate of 10-12 SCFM until a LOD value of ˜1% was achieved. The material was discharged from the system and analyzed for ibuprofen content uniformity.

Ibuprofen Content Uniformity Analysis:

The powder was poured on a flat surface and divided in six parts. A powder samples of about 50 mg, was taken form each part and placed in a separate flask. 25 mL of phosphate buffer pH 7.2 was added to each sample. The vials were shaken periodically. After 24 hours samples were taken from each vial, filtered through a 45 μm nylon filter and then analyzed by UV-Vis. The amount of ibuprofen in each sample was calculated by employing the UV absorption at λ=221 nm, the wavelength of maximum absorbance for ibuprofen. Standards were prepared in phosphate buffer pH 7.2. The content uniformity was very good, with a % RSD of 4.1.

### Example 10

### Preparation of a Blend of 1% Ibuprofen in Support Excipient by Blending in a V-Blender

99.0 g of support excipient were mixed together with 1.0 g of ibuprofen in a V-blender for two hours. The blend was discharged and analyzed for ibuprofen content uniformity using an equivalent method with the one described in Example 4. The content uniformity gave a % RSD of 15.99.

### Example 11

### Preparation of a Blend of 1% Hydrochlorothyazide in Support Excipient by Geometric Dilution

1 kg of a blend consisting of 10 g HCTZ, 987.5 g support excipient and 2.5 Magnesium stearate was prepared by geometric dilution as follows:

a. 10 g HCTZ and 90 g of support excipient were mixed and passed through a 40 mesh sieve. The mix was transferred to a glass container which was attached to the lab V-blender and blended for 10 minutes.

b. The blend obtained in step “a” was transferred together with another 400 g of support excipient to a 4 quart V-blender and blended for 15 min.

c. The blend obtained in step “b” was transferred together with another 497.5 g support excipient to an eight quart V-blender and blended for 15 min. Magnesium stearate was passed through a 40 mesh sieve and was added, in the V-blender, to the HCTZ/support excipient. This step was followed by 2 min blending. Samples were randomly selected from the blend for HCTZ content uniformity analysis.

HCTZ Content Uniformity Analysis:

The procedure employed to analyze the HCTZ content uniformity was in accordance with USP/NF procedure for Assay of “Hydrochlorothiazide tablets”. The content uniformity gave a % RSD of 13.55.

### Example 12

### Tableting by Direct Compression of a) 1% Chlorpheniramine Maleate Spray Coated on the Support Excipient, and b) 0.1% Chlorpheniramine Maleate Spray Coated on the Support Excipient

500 mg tablets were prepared by direct compression using a manual Carver Press, a 13 mm die and a 3000 lbs compression force. The obtained tablets were tested for hardness using a Varian, Benchsaver Series, VK 200 tablet hardness tester. The disintegration tests were performed with a Distek Disintegration System 3100, using 900 mL deionized water at 37±0.5° C. The tablet harndess and the disintegration times are listed in the table below.

| | | Disintegartion |
|---|---|---|
| Tablet Composition | Hardness (kp) | time (sec) |
| 1.0% Chlorpheniramine maleate spary | 23.5 | 40 |
| coated on support excipient | | |
| 0.1% Chlorpheniramine maleate spray | 20.3 | 47 |
| coated on support excipient | | |

1. A composition comprising:
a) a support excipient comprising:
about 75% to about 98% microcrystalline cellulose;
about 1% to about 10% at least one binder; and
about 1% to about 20% at least one disintegrant; wherein the microcrystalline cellulose, binder and disintegrant are indistinguishable when viewed with a SEM, thereby forming substantially homogeneous, substantially spherical particles of the support excipient; and
b) an API spray coated on the support excipient, wherein the API is about 0.01% to about 5% by weight of the support excipient.

2. The composition of claim 1 wherein the binder includes hydroxypropyl methylcellulose and the disintegrant includes cross-linked polyvinylpyrrolidone.

3. The composition of claim 1 wherein the support excipient comprises:
about 80% to about 90% microcrystalline cellulose;
about 2% to about 8% at least one binder; and
about 3% to about 12% at least one disintegrant.

4. The composition of claim 1 wherein the support excipient comprises:
about 85% to about 93% microcrystalline cellulose;
about 2% to about 5% at least one binder; and
about 10% at least one disintegrant.

5. The composition of claim 1 wherein the support excipient is formed by spraying an aqueous slurry comprised of the microcrystalline cellulose, binder and disintegrant.

6. A method of making a low dose API tablet, the method comprising:
a) forming a support excipient by
mixing a MCC slurry with a disintegrant slurry to form a MCC/disintegrant slurry;
mixing a binder in water to form a viscous binder slurry;
homogenizing the binder slurry with the MCC/disintegrant slurry to form a homogenized slurry; and
spray dry granulating the homogenized slurry to form substantially homogeneous, substantially spherical particles of support excipient;
b) spray coating the support excipient with about 0.01% to about 5% API; and
c) drying the spray coated support excipient to form particles of API coated support excipient.

7. The method of claim 6 further comprising directly compressing the particles of API coated support excipient to form a tablet without wet granulation of the API onto the support excipient.

8. The method of claim 6 wherein the support excipient comprises:
about 75% to about 98% microcrystalline cellulose;
about 1% to about 10% at least one binder; and
about 1% to about 20% at least one disintegrant.

9. The method of claim 6 wherein the support excipient comprises:
about 80% to about 90% microcrystalline cellulose;
about 2% to about 8% at least one binder; and
about 3% to about 12% at least one disintegrant.

10. The method of claim 6 wherein the support excipient comprises:
about 85% to about 93% microcrystalline cellulose;
about 2% to about 5% at least one binder; and
about 10% at least one disintegrant.

11. The method of claim 6 wherein spray coating the support excipient comprises:
spray coating the support excipient with a solution of the API;
spray coating the support excipient with a slurry of a micronized API; or
spray coating the support excipient with a low solubility API solution containing an API dissolution aid.

12. The method of claim 6 wherein the binder includes hydroxypropyl methylcellulose and the disintegrant includes cross-linked polyvinylpyrrolidone.

13. The method of claim 6 wherein the API is about 0.01% to about 1%.

14. A low dose API pharmaceutical tablet comprising:
about 0.01% to about 5% of at least one active pharmaceutical ingredient; and
a support excipient of substantially homogeneous, substantially spherical particles including:
a) microcrystalline cellulose;
b) at least one binder; and
c) at least one disintegrant.

15. The tablet of claim 14 wherein the support excipient includes:
about 75% to about 98% microcrystalline cellulose;
about 1% to about 10% at least one binder; and
about 1% to about 20% at least one disintegrant.

16. The tablet of claim 14 wherein the support excipient includes:
about 80% to about 90% microcrystalline cellulose;
about 2% to about 8% at least one binder; and
about 3% to about 12% at least one disintegrant.

17. The tablet of claim 14 wherein the support excipient includes:
about 85% to about 93% microcrystalline cellulose;
about 2% to about 5% at least one binder; and
about 10% at least one disintegrant.

18. The tablet of claim 14 wherein the binder includes hydroxypropyl methylcellulose and the disintegrant includes cross-linked polyvinylpyrrolidone.
